(19) United States
(12) Patent Application Publication
(10) Pub. No.: US 2014/0336515 A1
(43) Pub. Date: Nov. 13, 2014

(54) ULTRA-WIDEBAND AND INFRA-RED MULTISENSING INTEGRATION

(71) Applicant: ALBATROSS BREAST CANCER DIAGNOSTIC LTD, Tel Aviv (IL)

(72) Inventors: Shlomo Tangy, Rehovot (IL); Yosi Tangy, Rehovot (IL)

(73) Assignee: ALBATROSS BREAST CANCER DIAGNOSTIC LTD, Tel Aviv (IL)

(21) Appl. No.: 14/355,583

(22) PCT Filed: Nov. 4, 2012
(86) PCT No.: PCT/IL12/50440

§ 371 Date: May 1, 2014

Related U.S. Application Data
(60) Provisional application No. 61/555,015, filed on Nov. 3, 2011.

(51) Int. Cl. A61B 5/00; A61B 5/05; A61B 5/01

(52) U.S. Cl. CPC A61B 5/0035; A61B 5/015; A61B 5/0075; A61B 5/7264; A61B 5/05; A61B 2576/00
USPC 600/474

(57) ABSTRACT

A system for transition detection, including a thermal imaging device to form a thermal image of a subject positioned in a selected position, an ultra-wideband short range radar to scan the area of the subject identified in the thermal image while the subject is positioned in the selected position, a registration unit to combine information from the thermal image and the radar to form an enhanced image that provides indications of transitions inside the body of the subject.

### TECHNICAL FIELD

The present disclosure relates generally to the detection of transitions between layers, lesions or tumors in a living body and more specifically to performing the detection using an ultra-wideband radar in combination with an infra-red imaging system.

### BACKGROUND

A thermal imaging camera can provide an accurate image of an area on a living body with a temperature indication for each point on the image. The temperature indication may signify denser or less dense areas and provide indication of areas with increased blood flow or decreased blood flow. Currently a thermal image camera may be implemented as an integrated circuit for example using focal plane arrays (FPAs) that respond to longer wavelengths (mid and long-wavelength infrared). Typically the optic field of view (FOV) of an IR-FPA can differentiate between an area with a size as small as 1-10 mm², for example when using an 80 degree FOV. The use of thermal imaging in detecting tumors or other sub-dermal transitions can help in the location of suspect areas, for example to detect breast cancer.

In addition to using a thermal imaging device other methods may be used to verify the results. Tumors have different electrical properties from healthy surrounding tissue. Detecting those differences enables the detection of cancerous tissues.

Nowadays CMOS technology can be used to form integrated circuits that function with cycle speeds of tens to hundreds of GHz (ultra-wide band (UWB)). This ability enables the manufacture of integrated chips to operate a short range radar, which can be used for medical purposes to detect sub-dermal transitions. Typically an UWB radar implemented, for example as a 24 nm CMOS chip can provide sample rates for example of 50-100 GHz. The UWB radar can be designed to transmit short impulses instead of bursts over a carrier wave, for example impulses with pulse durations of 30-40 pico-seconds. The resulting wavelengths can be of a few millimeters (e.g. 5-10 mm) thus enabling the detection of transition with high resolutions for example differentiating between areas having a size as small as 1-10 mm².

The combination of the results from multiple imaging systems could increase the accuracy of the results, yet combining the results is generally a challenge for a practitioner. Typically equating the findings from two images requires that the images be taken of an identical area and even then the results are not easily equated since the internal tissue and blood vessels of the subject may be positioned differently relative to each other when taking each image (e.g. by a few millimeters) thus effecting the compatibility of the measurements. Additionally, the resolution of different imaging devices differs making it hard to equate the results.

In addition to the dielectric and thermal measurements, 3D UWB CMOS multi static radar-is implemented by the UWB radar.

UWB for Image Acquisition is an imaging technique that captures high resolution, 3D images through the use of harmless radio waves. An array of small antennas—similar in size to those inside a mobile phone—is placed around the part of the body to be imaged, such as a breast. The signal is transmitted from each element in turn and is then received by all the other elements, effectively ‘sweeping’ the imaged part of the body. The dielectrical differences between the various types of a tissue cause reflections from each border between tissues. Analyzing these reflections enables the calculation of a 3D image which can demonstrate tumors as small as a few millimeters across—dependent on the radar resolution. Unlike mammography, UWB RADAR does not require breast compression, making the whole process far more comfortable. The transmitted radio wave radar signal has non-ionizing energy and a power of less than cellular power—far below the safety threshold.

FUZZY LOGIC METHOD—and history collection.

The high resolution measurement of raw data, such as temperature, permeability and conductivity enables accurate decisions raw by new mathematical and statistical methods, such as fuzzy logic—taking into account the following parameters.

Position sensor aspects (FPA-matrix 256×256 for example and multi matrix/ Multi radar)

History of the measurements.

Scheduler time (since the temperature and the breast change periodically)

Decision taken in raw data from orthogonal sensors and multiple time is by definition has less false detection and better probability of detection.

### SUMMARY

An aspect of an embodiment of the disclosure relates to a system and method for non-invasive detection of transitions in the body of a subject. The detection of transitions enables a practitioner to locate abnormal situations, for example tumors or lesions. The transition detection is performed by positioning the subject in proximity to the system, which includes a thermal imaging device and an ultra-wideband short range radar. The thermal imaging device is used to form a thermal image of an area on the body of the subject and the radar is used to scan the same area either simultaneously or sequentially. The thermal image provides depth information by providing a temperature value for each pixel of the image. The radar provides depth information based on identifying tissue transitions, for example from dense tissue to less dense tissue or vice versa. The transitions cause a transmitted signal to be partially reflected causing the return of a signal to a receiver of the radar. Information about the position of transitions can be achieved by analysis of the returned signals. Optionally, the information from the radar scan can also be used to determine conductivity and/or permeability of regions in the subject's tissue. The thermal image and the information from the radar are provided to a registration unit that combines the information to form an enhanced image. Optionally, the registration unit may also map out the various regions in the subject's tissue (e.g. based on conductivity, permeability, or density) and of transitions between regions. The identification of the regions can be used to perform back reconstruction to locate the source of the temperature readings identified by the thermal image.

In an exemplary embodiment of the disclosure, the scan area is determined from analysis of the thermal image and notifying the radar where to scan. Alternatively, the scan area is a constant that is fixated by the geometry of the system, which requires the subject to be positioned in a specific way, which meets the requirements of the thermal imaging device and the radar. In an exemplary embodiment of the disclosure, the system first takes a thermal image and then based on the boundaries of the subject in the thermal image scans the subject with the radar. Alternatively, the thermal image and the radar commence simultaneously or the radar scan may precede acquisition of the thermal image.

In an exemplary embodiment of the disclosure, the pixel resolution of the thermal image is of a similar resolution as the radar scan. Alternatively, the radar scan is of a higher or lower resolution than the thermal image.

In an exemplary embodiment of the invention, multiple radars are operated simultaneously to control the scan direction. Alternatively, a single radar with multiple transmission antennas is used to control the scan direction. Likewise, reception may be carried out using one or more reception antennas.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a transition detection device, according to an exemplary embodiment of the disclosure;

FIG. 2 is a schematic illustration of a transmitted signal and received signal, according to an exemplary embodiment of the disclosure;

FIG. 3 is a flow diagram of a method of high resolution transition detection, according to an exemplary embodiment of the disclosure;

FIG. 4 is a schematic illustration of a UWB radar, according to an exemplary embodiment of the disclosure;

FIG. 5 is a schematic illustration of a processor for post processing an analyzed received signal, according to an exemplary embodiment of the disclosure;

FIG. 6 is a schematic illustration of multiple processing circuits connected to a central processor, according to an exemplary embodiment of the disclosure;

FIG. 7 is a schematic illustration of a received signal, according to an exemplary embodiment of the disclosure;

FIG. 8 is a schematic illustration of an accumulator circuit for recording threshold crossings by a received signal at multiple time intervals, according to an exemplary embodiment of the disclosure;

FIG. 9 is a schematic illustration of a wideband sampler circuit, according to an exemplary embodiment of the disclosure; and,

FIG. 10 is a schematic illustration of a grid representing scanned tissue of a subject having various regions, according to an exemplary embodiment of the disclosure.

### DETAILED DESCRIPTION

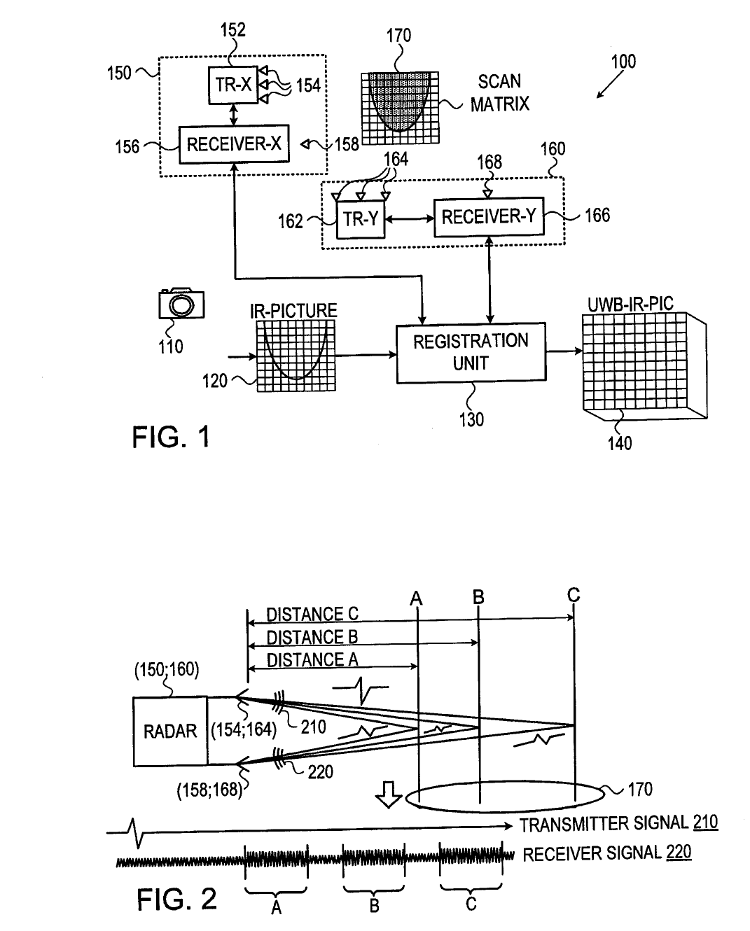

In an exemplary embodiment of the disclosure, an infra-red thermal imaging device is combined with a UWB short wave radar to simultaneously process a subject and detect sub-dermal transitions and/or identify regions, for example resulting from cancerous tumors. FIG. 1 is a schematic illustration of a transition detection device 100, according to an exemplary embodiment of the disclosure.

In an exemplary embodiment of the disclosure, transition detection device 100 includes an infra-red thermal imaging device 110 for taking a thermal image 120 of an area of the body of subject 170 to form an infra-red (IR) picture. Optionally, the thermal image 120 is represented by a matrix of pixels stored for example in a random access memory (RAM) or other type of memory device. In an exemplary embodiment of the disclosure, the resolution of the image may be user selected or constant, for example 320 by 240 or 256 by 256 pixels. Optionally, each pixel is represented by a value indicating a temperature level for the pixel.

In an exemplary embodiment of the disclosure, transition detection device 100 includes one or more radars designed to control a transmission signal. In some embodiments of the invention, a single radar with multiple antennas may be used to scan the subject 170. Alternatively, two radars may be used transmitting in orthogonal directions, for example as shown in FIG. 1 radar 150 transmits and receives signals in the X direction and radar 160 transmits and receives signals in the Y direction, perpendicular to radar 150. Optionally, by transmitting simultaneously from both radars (150, 160) a signal is focused to scan a two dimensional area. In an exemplary embodiment of the disclosure, the radars are programmed to scan the same area as sampled by the thermal imaging device 110. Optionally, each radar includes a transmission control (152, 162) coupled to a plurality of antennas (154, 164) to control the transmission of signals. In an exemplary embodiment of the disclosure, each radar (150, 160) includes a reception control (156, 166) coupled to one or more reception antennas (158, 168) to accept signals that are returned responsive to the transmissions of the radars (150, 160).

In some embodiments of the disclosure, a registration unit 130 provides details from the thermal image 120 to the radars to assure accurate scanning of the subject in the same boundaries as recorded in by the image, for example the registration unit 130 may provide the coordinates of the outline of subject 170. Alternatively or additionally, transition detection device 100 is constructed as a unit such that the radars (150, 160) are configured to scan substantially the same area as recorded by the thermal image 120. It should be noted that recordation of the thermal image 120 and scanning the subject 170 with the radars (150, 160) are performed within a short period of time, for example within less than a second or even a millisecond or less so that the subject can essentially be considered to be stationary for both processes. In some embodiments of the disclosure, the actual process may be performed sequentially or in parallel (e.g. taking the thermal image 120 while scanning the subject 170 with the radars (150, 160) or first taking the thermal image 120 and then scanning within the outline provided by the thermal image 120 or vide versa).

The thermal image 120 provides an indication of the internal layers behind each pixel by the temperature value recorded for each pixel of the thermal image 120, since the heat expansion coefficients of the human body are generally known and measured off-line in specific tests for our detection system. Accordingly, the thermal image 120 provides an indication of a position of interest below the surface, for example a cancerous tumor in a breast. Typically the resolution of the thermal image 120 may allow differentiation between areas of about 1-10 mm².

Likewise, as will be explained below in more detail, the radar scanning of the two dimensional area captured by the thermal image 120 provides an indication of the layers beyond the surface of the body of subject 170 being scanned. Optionally, within the same boundaries as the thermal image 120 the radars (150, 160) repeatedly transmit a pre-selected pulse for a specific duration and collect at receivers 156, 166 any available signals responsive to the transmissions. In an exemplary embodiment of the disclosure, the received signals are processed by comparing them to the transmitted signals to determine the thickness of layers, transitions between layers, material density, permeability and conductivity of the tissue in the volume below the surface of the body of subject 170. In an exemplary embodiment of the disclosure, these determined values can be used to identify the heat expansion coefficients for various regions in the volume thus enabling calculation of the position of a volume serving as a heat source and the heat flow path leading to the thermal image measurements. Typically, volumes serving as heat sources provide an indication of enhanced blood flow, which may be related to cancerous activity.

In an exemplary embodiment of the disclosure, the scanning is performed by transmitting signals of varying frequencies and/or of varying phase from different antennas of the radars (150, 160) to scan the body of the subject within the selected boundaries and achieve measurements with a resolution close to the resolution of the thermal image 120. However it should be noted that due to the physical difference of the acquiring methods the measurements from a radar scan and from an infra-red thermal imaging device 110 do not provide images with a one to one pixel match. Optionally, the results from thermal image device 110 and the results from the radars (150, 160) are provided to registration unit 130 that combines the results and forms an UWB-IR combined image 140 with enhanced transition detection. In some embodiments of the disclosure, the UWB-IR image 140 may be a representation of a three dimensional image and/or may include a list of coordinates of detected transitions with an indication relating to the sharpness of the change.

In an exemplary embodiment of the disclosure, registration unit 130 may first correlate between the images from their centers and then combine outward toward the boundaries. Alternatively or additionally, other known methods of combining images may be used.

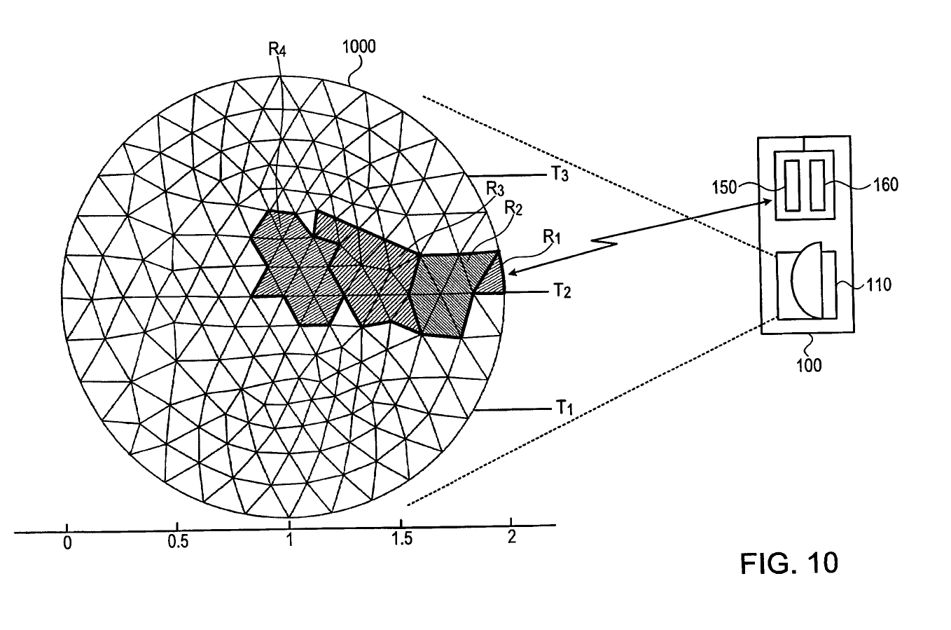

FIG. 10 is a schematic illustration of a grid 1000 representing scanned tissue of a subject having various regions (e.g. R1, R2, R3, R4), according to an exemplary embodiment of the disclosure. Optionally, radars (150, 160) identify regions of common density or permeability or conductivity and transitions thereof. Optionally, infra-red thermal imaging device 110 identifies various temperatures (T1, T2, T3) on the surface of the body of the subject. By calculating heat expansion coefficients for the regions from the surface inward the regions serving as the origin of the measured temperature can be identified.

In an exemplary embodiment of the disclosure, the elements of device 100 (thermal imaging device 110, radars (150, 160), and registration unit 130) are implemented as integrated circuits, for example using CMOS technology. Optionally, some elements may be implemented as separate integrated circuits and electrically and/or physically coupled to each other or all the elements may be incorporated into a single integrated circuit to prevent interference resulting from the transition of electrical signals from different integrated circuits.

FIG. 2 is a schematic illustration of a transmitter signal 210 and receiver signal 220, according to an exemplary embodiment of the disclosure. In an exemplary embodiment of the disclosure, radar (150, 160) transmits transmitter signal 210 toward subject 170. Subject 170 is exemplified by 3 transition layers (A, B and C), for example when transmitter signal 210 coincides with the surface of the body of subject 170 (layer A) part of the signal continues forward and part is returned to be detected as part of receiver signal 220. Likewise a tumor, blood vessel or muscle having a different density than the layer before it may also serve as a transition layer (e.g. B and C) causing part of transmitter signal 210 to continue and part to be returned. Optionally, transmitter signal 210 is represented by a short pulse (e.g. of 10-20 picoseconds) followed by a pause so as not to hinder receiver signal 220.

In an exemplary embodiment of the disclosure, radars (150, 160) transmit signals from the antennas (154, 164) with different phase shifts so that different positions in subject 170 within the selected boundaries will be scanned to form an image of the entire volume being examined. Optionally, each phase shift may be considered to correlate to a specific pixel location of thermal image 120 although the measurement accuracy may differ from that of the thermal image 120. Optionally, for each selected phase shift a signal of a specific time duration and pattern is transmitted forming transmitter signal 210. Optionally, transmitter signal 210 is pre-selected as specific pulse patterns to simplify analysis of receiver signal 220. In an exemplary embodiment of the invention, the phase shifts may be selected to cause the scanning to correlate to scanning rows and/or columns of the pixel locations of thermal image 120.

In an exemplary embodiment of the disclosure, while scanning an area a specific sequence of pulses may be used for each scan row or for each column to allow the receiver to differentiate between rows or columns based on the receiver signal 220. Optionally, specific radar codes may be used, for example the Barker code. In some embodiments of the disclosure, the transmitter signal 210 is repeated a number of times (e.g. 10-100) for each phase shift to increase accuracy of the measurements based on the receiver signal 220. Optionally, the pulse pattern may be repeated before moving to a new phase shift or the whole scanning sequence may be repeated multiple times (e.g. 10-100) providing a single pulse for each phase shift setting.

In an exemplary embodiment of the disclosure, receiver signal 220 is made up from signals returned from a transmitter signal 210 arriving at different transitions layers (A, B, C). Receiver signal 220 may also include noise or signals returned from transmitter signals 210 from adjacent pixel location. Typically the noise and signals from other locations will be weaker than the signals initially returned from transition layers (A, B, C). Optionally, by transmitting different types of pulses for transmitter signals 210 at different pixel locations it may be possible to differentiate between receiver signals 220 based on the transmitter signal 210 from which they originated.

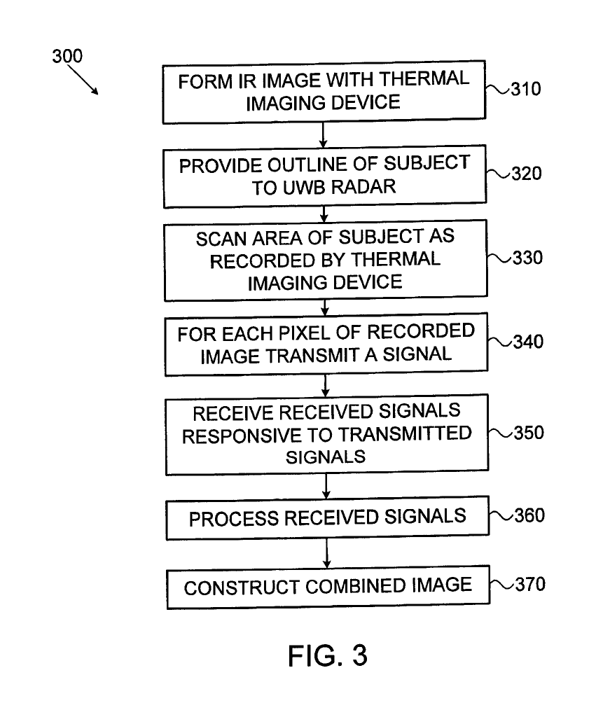

FIG. 3 is a flow diagram of a method of high resolution transition detection, according to an exemplary embodiment of the disclosure. In an exemplary embodiment of the disclosure, a thermal image 120 is recorded (310) of a selected area on the body of the subject 170. Optionally, the thermal image 120 is of a high resolution allowing differentiation between areas with a size as small as about 1-10 mm². Optionally, details of the dimensions of the subject 170 taken by the thermal image 120 are provided (320) to radars 150, 160 to scan the subject based on the pixels of the thermal image 120 with a transmitter signal 210. Alternatively, thermal image device 110 and radars 150, 160 may be synchronized to sample the same area without coordinating between them, for example requiring the subject to be positioned at a specific position relative to transition detection device 100. In an exemplary embodiment of the disclosure, radars 150, 160 synchronously scan (330) the same area of the subject 170 as recorded by thermal image device 110, for example by transmitting (340) transmitter signals 210 corresponding to each pixel of thermal image 120.

In an exemplary embodiment of the disclosure, radars 150, 160 receive (350) receiver signals 220 responsive to the transmitter signals 210. Optionally, the receiver signals 220 may result from reflections from different layers of the body of the subject 170 or from various sources of noise. In an exemplary embodiment of the disclosure, the radars 150, 160 process (360) the receiver signals 220 to determine where there are transitions in the third dimension (Z coordinate) of the scanned subject 170.

In an exemplary embodiment of the disclosure, registration unit 130 of transition detection device 100 constructs (370) a combined image based on the measurements of the thermal image device 110 and the measurements of the radars 150, 160. Optionally, the results may be displayed graphically on a computer as a three dimensional image or may be provided as tables of coordinates of transitions and level of sharpness of transition or other types of output.

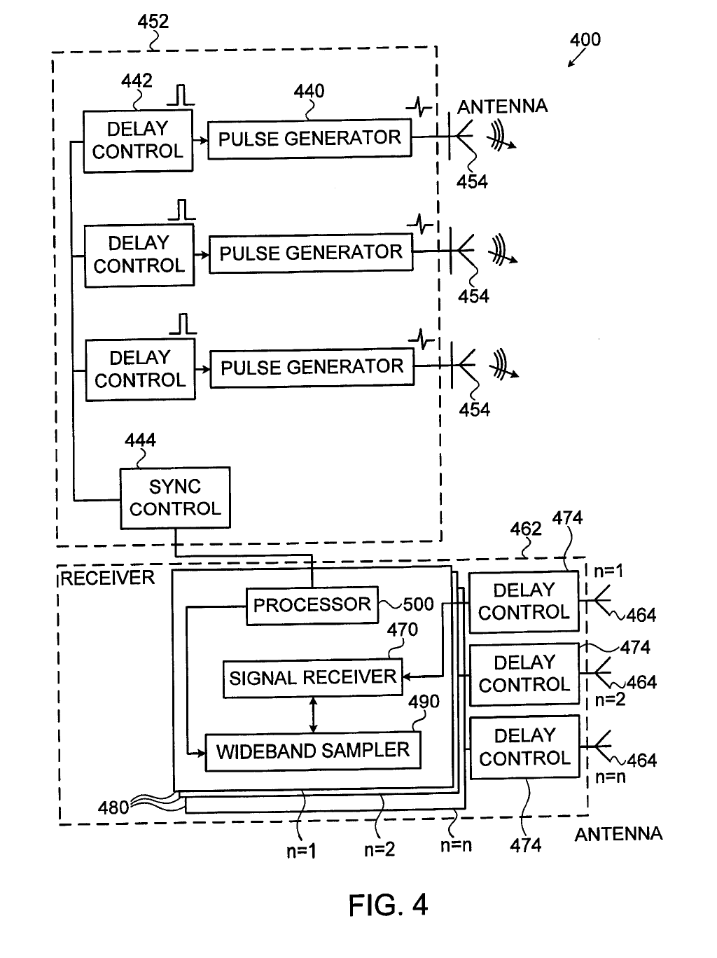

FIG. 4 is a schematic illustration of an UWB radar 400, according to an exemplary embodiment of the disclosure. In an exemplary embodiment of the disclosure, radar 400 includes a transmission control 452 and a reception control 462. Optionally, transmission control 452 includes a sync control 444 that controls the timing of radar 400 by synchronizing between the transmission control 452 and the reception control 462. In an exemplary embodiment of the disclosure, sync control 444 provides a signal to transmission control 452 to begin transmission and synchronously notifies reception control 462 to prepare to receive signals in sync with the transmissions. Accordingly, reception control 462 processes receiver signal 220 using the timing information of the transmitter signal 210 from which it originated.

In an exemplary embodiment of the disclosure, transmission control 452 includes one or more antennas 454 to be able to control the direction of the transmitted signal. Optionally, each antenna 454 is controlled by a delay control 442 that forms a time delay for each antenna 454 to control propagation of the combined transmission signals. In an exemplary embodiment of the disclosure, the delay control 442 provides a signal to a pulse generator 440 to generate a pulse and transmit it using an antenna 454.

In an exemplary embodiment of the disclosure, reception control 462 includes one or more antennas 464, each connected to a delay control 474 to adjust the timing of the reception signal for each antenna 464. In some embodiments of the disclosure, reception control 462 may have the same number of antennas 464 as transmission control 452 or it may have a different number of antennas 464. Optionally, if the reception control 462 has the same number of antennas 464 as the transmission control 452 then the delay control 474 of the reception control 462 may be in sync with the delay control 442 of transmission control 452.

In an exemplary embodiment of the disclosure, reception control 462 includes multiple processing circuits 480 (e.g. 1 to n), one processing circuit 480 for each antenna 464. Optionally, each processing circuit 480 includes a signal receiver 470 to receive the received signal 220 from antenna 464. Optionally, the received signal 220 is provided to a wideband sampler 490 that analyzes the signal to determine, the signal strength from each distance measured by its associated antenna 464. The results of the wideband sampler 490 are then provided to a processor 500 that filters and processes the results derived from the associated antenna 464.

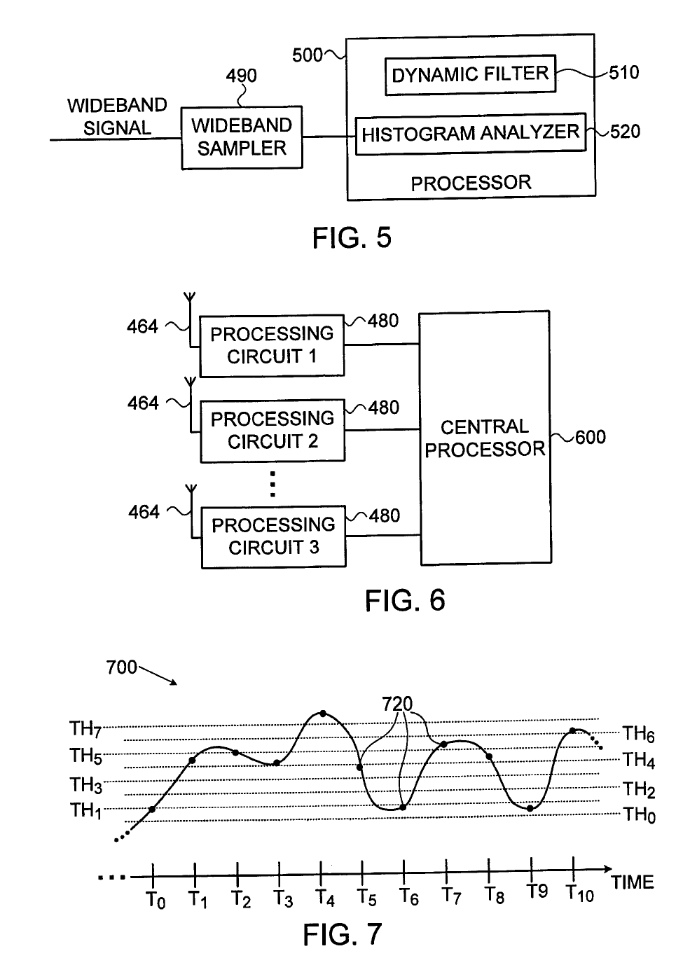

FIG. 5 is a schematic illustration of processor 500 for post processing the analyzed received signal 220, according to an exemplary embodiment of the disclosure. Optionally, processor 500 includes a dynamic filter 510 and histogram analyzer 520. In an exemplary embodiment of the disclosure, after analyzing receiver signal 220 from each antenna 464 and determining the signal strength from each distance, the results are reviewed by dynamic filter 510 to correct analysis errors related to frequency bands, phases and amplitudes of the received signal. Optionally, dynamic filter 510 may amend the results to correct such errors. Likewise histogram analyzer 520 checks for logical errors in the results and may discard or add values to form continuous and meaningful results. Optionally, the post processing is performed on each received signal so that the results may be combined.

FIG. 6 is a schematic illustration of multiple processing circuits 480 connected to a central processor 600, according to an exemplary embodiment of the disclosure. Optionally, the results of processing receiver signals 220 from each processing circuit 480 are provided by each local processor 500 to a central processor 600 that combines the results and provides them to registration unit 130. In an exemplary embodiment of the disclosure, central processor 600 can instruct radar 400 to repeat the scanning and calculations if the results are not as expected or non decisive, for example if the results indicate that the subject moved or that electronic interference may have caused the results to be unsatisfactory. In some embodiments of the disclosure, the central processor 600 may be an external general purpose computer. Alternatively, central processor 600 may be located in registration unit 130 or as part of radar 400.

FIG. 7 is a schematic graph of a received signal 700, according to an exemplary embodiment of the disclosure. In an exemplary embodiment of the disclosure, the moment that transmission control 452 transmits a transmission signal 210 is marked as T₀. From T₀ until a pre-selected time (e.g. T₁₀) at specific time intervals receiver processing circuit 480 samples receiver signal 220 and records a signal strength 720 of receiver signal 220. In an exemplary embodiment of the disclosure, the time intervals represent the distance traveled by the transmitted signal 210 from transmission antenna 454 toward subject 170 and then reflected back toward receiver antenna 464. Graph 700 shows the signal strength 720 at each time interval. Optionally, processing circuit 480 samples receiver signal 220 until a pre-selected time Tₙ (e.g. T₁₀), at which the received signal 220 is expected to be returned from positions which are at a distance, which is not of interest, for example from a distance that is beyond the dermal layer of subject 170 or from a distance relative to the transmission antenna 454 that is known to provide results that are too weak to process.

In some embodiments of the disclosure, the results in graph 700 represent the measurements of a receiver signal 220 from a single transmitted pulse aimed at a specific location. Alternatively, the results in graph 700 may represent measurement of multiple pulses transmitted sequentially to a specific location, for example a sequence of 10-100 pulses transmitted one after another toward the same location, between time T₀ and the pre-selected time Tₙ. Optionally, when transmission control 452 focuses the transmissions to a new location, sync control 444 notifies processing circuit 480 to pass on the reception measurements and to start counting from T₀ again for the new location. Alternatively, the measurements may be performed for a whole row or column of the scanned area.

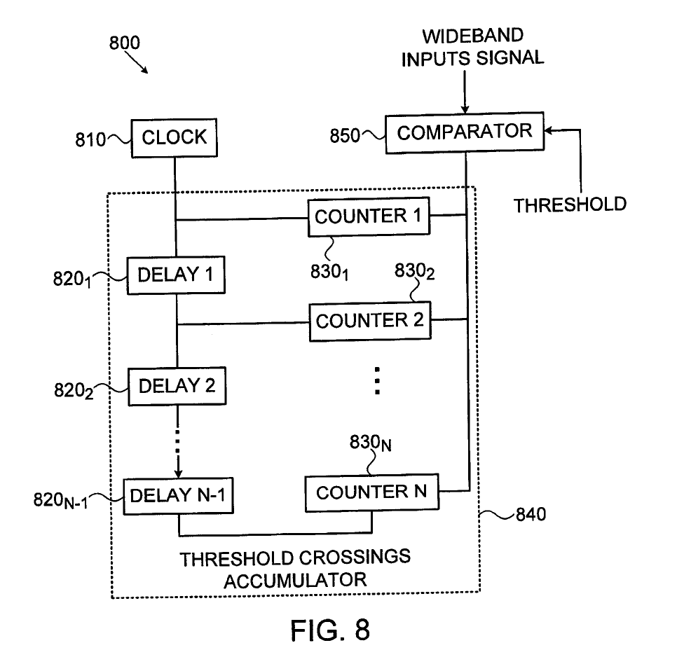

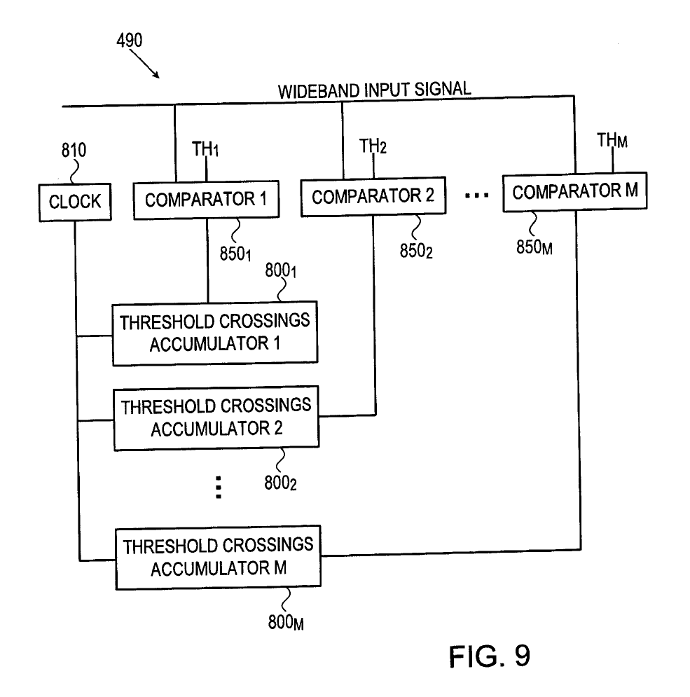

FIG. 8 is a schematic illustration of an accumulator 800 for recording threshold crossings by receiver signal 220 at multiple time intervals, and FIG. 9 is a schematic illustration of wideband sampler 490 that includes multiple accumulators 800, one for each threshold level, according to an exemplary embodiment of the disclosure. In an exemplary embodiment of the disclosure, wideband sampler 490 is designed as an integrated circuit that receives a signal, for example receiver signal 220, such as depicted in graph 700 and forms a matrix of counters 830ᵢ in accumulators 800ⱼ. The values of the matrix represent the details depicted in graph 700: for each time interval (T₀ to time Tₙ) at each threshold value (TH₁ to THⱼ), counter 830ᵢ₊₁ of accumulator 800ⱼ adds a 1 if the threshold value at that time interval Tᵢ is greater or equal to (i.e. crossed over) the threshold value.

Optionally, as receiver signal 220 is received, a clock 810 sets the timing and at each time interval Tᵢ wideband sampler 490, compares all the threshold levels in parallel using comparators 850ⱼ, with the signal. The results of the parallel comparison are recorded in accumulators 800ⱼ in a respective counter 830ᵢ₊₁ according to the time interval. In an exemplary embodiment of the disclosure, each accumulator 800ⱼ includes a cascade of delay elements 820ᵢ to enable the correct counter 830ᵢ₊₁ to record the result of the comparison based on the time interval. Optionally, the counting process is repeated for each pulse transmitted as a transmitter signal 210 from transmission antenna 454 until transmission control 452 updates the direction of transmitter signal 210 (e.g. by changing the signal phase on some of the antennas 454). Optionally, a single transmitter signal 210 may include multiple pulses, for example 100-1000 pulses. In an exemplary embodiment of the disclosure, when the direction of transmission is changed the results in counters 830ᵢ are passed on to processor 500 to be processed. Alternatively, the results may be accumulated for each scan row or each scan column.

In an exemplary embodiment of the disclosure, comparing the receiver signal 220 to all the threshold values in parallel instead of sampling the value of the receiver signal 220 and using an analog to digital converter allows determination of the signal strength in a single step. As a result the processing speed is increased so that the radar may function at cycle speeds of 50-100 GHz with minimal delay. Alternatively, the receiver may use buffers to store the received signal and use a standard analog to digital converter to sample the received signal and process the signal at a slower pace, however that would delay the response from transition detection device 100.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure. It will also be appreciated by persons skilled in the art that the present disclosure is not limited to what has been particularly shown and described hereinabove.

We claim:

1. A system for 3D transition detection, comprising:
a) at least two sensors selected from a group consisting of a temperature sensor, permeability sensor and conductivity sensor implemented on CMOS chip technology;
b) a processing unit utilizing a fuzzy logic mathematical and statistical algorithms for generating transition decisions based on data obtained from the at least two sensors.

2. A system for transition detection, comprising:
a thermal imaging device to form a thermal image of a subject positioned in a selected position; an ultra-wideband short range radar to scan the subject positioned in the selected position and form an image; wherein the area of the image formed by the radar scan and by the thermal imaging device is substantially the same;
a registration unit to combine information from the thermal image and the radar scan to form an enhanced image that provides indications of transitions inside the body of the subject.

3. A system according to claim 2, wherein the thermal imaging device and the radar operate simultaneously.

4. A system according to claim 1, wherein the thermal imaging device and the radar operate sequentially.

5. A system according to claim 1, wherein the radar comprises multiple transmission antennas to control the direction of the transmission signal.

6. A system according to claim 1, wherein the radar comprises multiple reception antennas to enhance reception.

7. A system according to claim 1, wherein the system comprises multiple radars to control the transmission signal.

8. A system according to claim 7, wherein at least one of the radars transmits orthogonally relative to the others.

9. A system according to claim 1, wherein the radar scan identifies in the body of the subject regions of common density or permeability or conductivity and transitions from one region to another.

10. A system according to claim 9, wherein the system determines heat expansion coefficients for the regions and identifies regions which are relatively hotter than others based on the results of the thermal image.

11. A system according to claim 1, wherein the resolution of the thermal image is user selectable.

12. A system according to claim 1, wherein said indications include a three dimensional image.

13. A system according to claim 1, wherein said indications include a list of coordinates of transitions and a level of sharpness of the transition.

14. A system according to claim 1, wherein said system is incorporated into a single integrated circuit.

15. A system according to claim 1, wherein said radar transmits different pulse patterns when focused to different locations.

16. A system according to claim 1, wherein said radar transmits the same pulse pattern multiple times when focused to a specific location.

17. A system according to claim 1, wherein said radar includes a synchronization control that synchronizes between transmission and reception of radar signals.

18. A system according to claim 1, wherein said radar includes a reception unit to receive transmitted signals; and
wherein the reception unit samples the received signals periodically to determine a propagation time of the signal from the radar and back and a signal strength of the signal.

19. A system according to claim 18, wherein determination of the signal strength at a specific propagation time is performed by comparing the sampled signal in parallel to multiple threshold values to determine the value in a single step.

20. A method of transition detection, comprising:
forming a thermal image of a subject positioned in a selected position;
scanning substantially the same area of the subject appearing in the thermal image with an ultra-wideband short range radar while the subject is positioned in the selected position;
transferring the thermal image and the results of the radar scan to a registration unit;
combining information from the thermal image and the results of the radar scan to form an enhanced image that provides indications of transitions inside the body of the subject.

21. A method according to claim 20, further comprising determining the area of the subject to scan with the radar from the thermal image.

22. A method according to claim 20, further comprising determining from the radar measurements one or more of the following parameters related to layers inside the body of the subject: thickness, material density, permeability, and conductivity.

23. A method according to claim 22, further comprising identifying heat flow paths based on the determined parameters and calculating the location of volumes serving as heat sources in view of the values from the thermal image.
